(19) United States
(12) Patent Application Publication
(10) Pub. No.: US 2013/0222765 A1
(43) Pub. Date: Aug. 29, 2013

(54) CONTACT LENS OPTIMIZER

(71) Applicant: DigitalVision, LLC, (US)

(72) Inventors: Keith P. Thompson, Atlanta, GA (US); Jose R. Garcia, Mableton, GA (US)

(73) Assignee: DigitalVision, LLC, Atlanta, GA (US)

(21) Appl. No.: 13/766,429

(22) Filed: Feb. 13, 2013

Related U.S. Application Data
(60) Provisional application No. 61/598,147, filed on Feb. 13, 2012.

(51) Int. Cl. A61B 3/10

(52) U.S. Cl. CPC A61B 3/1015
USPC 351/211; 351/246

(57) ABSTRACT

Vision testing methods and apparatuses are disclosed, the methods including measuring the modulation to a wavefront of light imparted by a contact lens, determining the wavefront modulation necessary to emulate the optical properties of the lens as worn on a patient's eye, generating a static or dynamic image viewable by a patient, modulating the wavefront of the image remote from the patient to attain the wavefront necessary to emulate the optical properties of the lens as worn on a patient's eye, and relaying the wavefront to a plane nearby, on, or within the patient's eye. The apparatuses include devices for measuring the modulation to a wavefront of light imparted by a contact lens, determining the wavefront modulation necessary to emulate the optical properties of the lens as worn on the patient's eye, generating a static or dynamic image viewable by a patient, modulating the wavefront of the image remote from the patient to attain the wavefront necessary to emulate the optical properties of the lens as worn on the patient's eye, and relaying the wavefront to a plane nearby, on, or within the patient's eye.

### BACKGROUND OF THE DISCLOSURE

1. Field

Disclosed is a method and apparatus of simulating the optical properties of one or more contact lenses as worn on the patient's eye under real-world conditions at far away, close, and intermediate distances, and under monocular or binocular viewing conditions.

2. Description of the Related Art

The first known contact lens was fabricated and fit in the late 1800's. By the middle of the twentieth century, plastic lenses were devised and made smaller, thinner, and with designs that improved comfort and vision. Hard lenses remained difficult for many patients to wear and the first commercially available soft contact lens made of a water absorbing plastic known as hydroxyl-ethylmethacrylate (HEMA) was introduced by Baush & Lomb in 1971. The soft lenses were thinner and much more comfortable, allowing many more patients to become successful contact lens wearers. Today, approximately 90% of contact lenses sold in the United States are soft lenses.

Bifocal, or more precisely, multi-focal, contact lenses designed to provide corrections for near and far viewing distances for presbyopic patients first became available in 1982. Since their introduction, multi-focal contact lenses have undergone considerable improvement and there are now many different designs for multi-focal contact lenses available.

Toshida (Clinical Ophthalmology 2008:2(4) 869-877) classified multi-focal contact lenses into alternating or translating vision lenses and simultaneous vision lenses which can be further classified as refractive and diffractive lenses.

Translating vision lenses are also called segmented lenses in which distance and near corrections are provided in the upper and lower portions of the lens respectively. The lens is physically translated by the lower lid, thereby bringing the reading portion of the contact lens in line with the visual axis when the patient looks downward to read.

Simultaneous vision lenses of the refractive type can be categorized as the DeCarle type of lens in which the central and peripheral parts of the lens are used for distance and near vision respectively. In the Alges type of refractive lenses, these zones are reversed and the reading correction is provided in the central portion of the lens.

In refractive presbyopia-correcting contact lenses, the wearer sees both near and distance images and the brain optimizes the perceived image of the object of interest. One advantage to this type of lens is that it is generally free from the problem of lens rotation, but fitting and centering of the lens over the pupil are important.

Another type of refractive multi-focal lens is a non-spherical or aspheric type lens. In aspheric lenses, the posterior surface, or anterior and posterior surfaces are aspheric, and the central and paracentral portions provide distance and near vision, respectively. The power of the lens changes gradually between different portions of the lens.

Another type of refractive multi-focal lens is a design in which the optical center of the lens is shifted slightly nasally to approximate the visual axis of the eye, which in most patients, is inferior and nasal to the geometric center of the pupil. This type of lens is associated with good near vision.

In diffractive multi-focal contact lenses there are concentric grooves on the posterior surface which cause diffraction of the light, much like the design of a Fresnel lens. The central portion of a diffractive contact lens often is designed to provide good distance vision, while the diffractive zone provides near focus. A disadvantage to diffractive designs is that poor contrast and glare are often problems.

In addition to these multi-focal contact lenses, it is possible to fit patients with monovision contact lenses. In a monovision fit, the doctor typically provides the patient with a distance-correcting contact lens in the dominant eye and a near-correcting contact lens in the fellow eye, although these may be reversed in certain patients. The patient learns to adapt to monovision, and some patients may function at all distances without additional corrections.

There are also lenses designed to provide modified monovision such as the Frequency 55 Multifocal by Coopervision that is equipped with a D-lens for distance vision in the center for the dominant eye and an N-lens with near vision in the center for the non-dominant eye as well as the UltraVue™ 2000 Toric Multifocal equipped with the D-lens (distant vision) and an N-lens (near vision) in the center and a toric design for the posterior part of the lens.

As more and more presbyopia correcting contact lenses become available, eye care professionals and their patients are exposed to an expanding number of choices and marketing claims that are difficult to evaluate objectively. Clinical experience with presbyopia-correcting-contact lenses has demonstrated that not all patients are good candidates for these lenses and are dissatisfied with their vision requiring the contact lenses to be replaced by a lens of different design. Trying on and replacing numerous lenses to attain satisfactory vision is inconvenient and costly for patients.

It is also known by those skilled in the art that contact lenses of the same nominal power have very different designs that provide different qualities of vision. FIG. 2 shows the power profile of 13 commercially available soft contact lenses measured by a commercially available lens mapping device. While each of the lenses shown in FIG. 2 has a nominal power of −3D, the peripheral power profiles of the lenses differs significantly. These differences in power will provide patients with different quality of vision as the pupil dilates. Those patients with large amounts of pre-existing spherical aberration will notice a substantial decline in night vision with the lens designs that do not correct, or that worsen, the spherical aberration in the periphery of the pupil compared to lens designs that provide for substantial correction of the spherical aberration. These lenses were tested with an expensive specialty optical instrument not available to most practitioners. Therefore, prior art means of fitting contact lenses fail to provide the practitioner with information needed to select the best corrective contact lens design for a particular patient.

Prior art methods do not provide the patient any means to preview, compare, and select the lens design, among a plurality of designs that will provide them with the best vision. For example, the lenses shown in FIG. 2 and FIG. 3 must be successively tried on in a sequential fashion. After one lens has been inserted, the quality of vision that it provides is observed, the lens is removed and a different lens inserted and the quality of vision that it provided observed. This sequential comparison method is a poor means of making an effective comparison of lens designs because they lenses cannot be compared simultaneously, on a side-by-side basis.

Measurement of the Optical Properties of Contact Lenses.

Methods to measure the optical properties of contact lenses are known. European Patents EP0129388A2 and EP1759167 teach methods and apparatuses for measuring soft and gas permeable contact lenses by using optical probe means. A commercially available instrument that can measure the optical properties of contact lenses is the ClearWave™ Contact Lens Precision Aberrometer manufactured by Lumetrics Corporation, Rochester, N.Y.

Spatially resolved refractometers are known, such as the device described by Webb in U.S. Pat. No. 6,000,800. In this disclosure, Webb teaches that a spatially resolved refractometer may be configured to determine the optical characteristics of an optical system such as a contact lens.

Contact Lens Simulators.

Multifocal contact lens simulators are known such as the device described in US Patent application US 2011/0080562. This disclosure teaches a multifocal contact lens simulator that includes an optical system that allows an object to be observed through it, and a test lens holder which holds a prescribed test contact lens. The contact lens holder is installed at a position optically conjugate with a position at which an eye of an observer is to be placed.

However, the prior art method of simulating the optical properties of contact lenses does not provide the patient a realistic assessment of the quality of vision that the contact lens will provide, because no means are provided to view objects of varying size, shape, color, contrast, and illumination; nor does the aforementioned patent application teach a means to view objects at near, intermediate, and far away distances, or permit testing under binocular conditions.

Prior art devices provide no clinically practical method for determining which, if any, of the available contact lens designs will provide a particular patient with a satisfactory level of visual function when wearing a contact lens of a given design, nor do they permit the patient to preview, compare, and select the contact lens design that they prefer based upon a comparison of the image producing properties of the contact lenses.

To address these unsolved problems, this disclosure teaches a new method and apparatus that permits patients to preview and compare the distant, intermediate, and near vision that a particular contact lens design will provide, and allow the patient to compare the vision provided by a plurality of designs while observing realistic images of real-world scenes over a variety of viewing distances. This provides patients the ability to preview, compare, and select the contact lens design that is most likely to provide satisfactory visual function before the lenses are dispensed.

### SUMMARY

A contact lens optimizer is disclosed. An optical device is provided that measures the modulations to the wavefront of an image that passes through a contact lens. A contact lens vision emulator is provided comprised of a viewing station, a wavefront generator and a focusing system.

In the wavefront generator, a projector, preferentially a digital display, projects a static or dynamic image through optical elements that are under control of a computer. A focusing system, preferentially a spherical field mirror, focuses the wavefront generator to a position that is optically conjugate to the patient's eye. The focusing system may provide a near-viewing display accessory and an eye tracker to stabilize the positions of the projected images.

The wavefront generator is then adjusted to produce an image on the patient's retina that emulates the image that would result if the contact lens to be emulated was worn on the patient's cornea.

The disclosure teaches the ability to test the tolerance of prospective contact lens patients to different contact lens designs over a range of different distances and viewing conditions. This permits contact lens emulation under natural conditions, void of obstructing instruments and other limitations that are inherent in the prior art, and it allows the patient to preview, compare, and select the contact lens design, from a plurality of designs, that will provide optimal eyesight with minimal visual side effects for the patient's particular visual needs.

### BRIEF DESCRIPTION OF THE DRAWINGS

FIG. 1 is a depiction of three different multifocal contact lenses of different designs A, B, and C, showing zones for distance vision D in light color and zones for near vision correction N in shaded color.

FIG. 2 is a depiction of the power profile of 13 commercially available monofocal contact lenses each having a nominal power of −3D in Diopters, as a function of the horizontal distance in mm from the center of the lens.

FIG. 3 is a depiction of the lenses in FIG. 2, showing the power profile of the right side of the lenses and the Zernicke coefficients of the wavefront error of the native eye and when wearing CIBA and Acuvue lenses.

FIG. 4 is a diagrammatical side elevational view of the patient chair and rear tower of the contact lens emulator.

FIG. 5 is a perspective view of the apparatus.

FIG. 6 is a partial top plan view of the wavefront generators for the right and left eyes with the adjustable optical elements removed.

FIG. 7 is a partial detailed view of an embodiment of the wavefront generator for the right eye with the adjustable lenses in position.

FIG. 8 is a table listing the identity of the adjustable lens elements shown in FIG. 7.

FIG. 9 is a block diagram of inputs and outputs of the System computer.

FIG. 10 is a diagrammatical side elevational view of a preferred embodiment of the invention in which the optical channels for each eye have two independent wavefront generators.

FIG. 11 is a diagrammatical front perspective view showing the near viewing accessory of the contact lens emulator.

FIG. 12 depicts the patients monocular right eye view of the field mirror and near viewing accessory while the contact lens optimizer is generating images that emulate images formed by different contact lens designs, thereby allowing the patient to preview, compare, and select the contact lens design that will provide the best vision following fitting.

### DETAILED DESCRIPTION

One embodiment of the apparatus has two components. A contact lens measurement means is used to characterize the optical properties of one or more contact lenses and to determine the modulation of the wavefront of an image that is necessary to reproduce or to emulate the optical properties of the contact lens once it is placed on the cornea of a patient's eye. The second component is a contact lens emulator means that recreates the optical properties of the contact lens for patient testing. In an alternative embodiment, the optical properties of the contact lens are provided elsewhere.

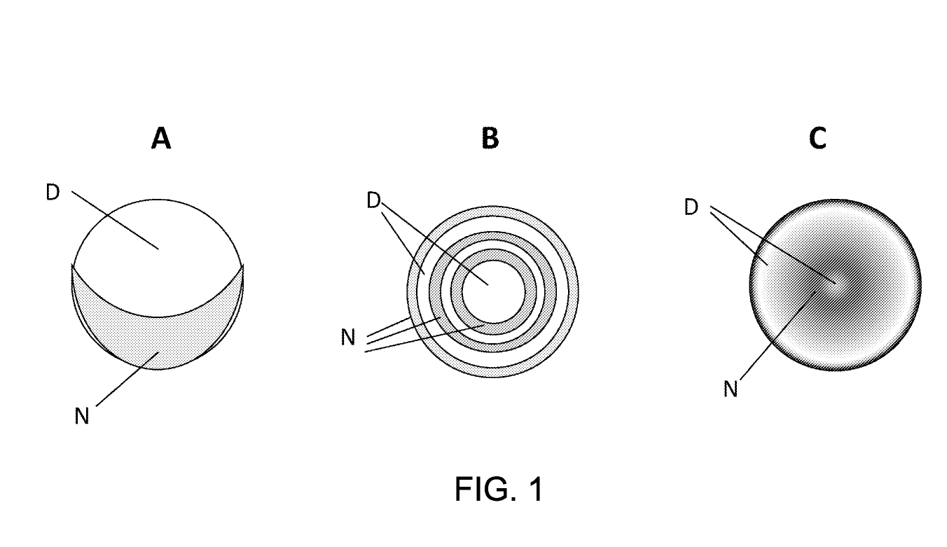

FIG. 1 shows three multi-focal contact lenses A, B, C that have different optical designs. Three lenses are shown for exemplary purposes to illustrate the three major types of presbyopia correcting contact lenses in use today; bi-focal, diffractive, and refractive. The instrument is not limited to emulating these types of designs and it may be used to emulate future designs that are developed. In addition, the instrument can be used to measure any number of designs of a given type but that vary in their design characteristics, dimensions, materials, and other properties, including single vision spherical and toric contact lenses. Optical characterization means, not shown, is used to characterize the optical properties of each contact lens, independently. Such optical characterization means suitable for use in this apparatus are known and may be provided by ClearWave™ Contact Lens Precision Aberrometer manufactured by Lumetrics Corporation, Rochester, N.Y. In other embodiments, optical characterization means may be a spatially resolved refractometer, a Schack-Hartmann wavefront sensor, or a similar device that uses optical probe means. In addition to measuring the phase change imparted to an image by the contact lens, the change in image intensity and/or image intensity as a function of wavelength can be measured by a suitable device such as a spectrometer. Following measurement with optical characterization means, the optical properties of the contact lens may be described by a mathematical function, for example, a Zernicke series, a Fournier transform series, or a Taylor expansion series. Those skilled in the art are familiar with these, and other mathematical functions, that may be used to describe the phase changes, or modulation to the wavefront of light, that occurs as light passes through the contact lens.

It is also known to those skilled in the art that the total phase change imparted to a wave of light by an contact lens is a function of both the shape of the front and back surfaces of the contact lens and the difference between the index of refraction of the contact lens and the index of refraction of the medium surrounding the contact lens. The index of refraction of a material is a ratio of the speed of light in a vacuum to the speed of light through the material. Because the contact lens is designed to be worn on the cornea and it is known that the tears that surround the contact lens have a refractive index of 1.33698, appropriate correction factors can be applied to accurately determine the optical properties of the contact lens when it is placed on the cornea even though the measurements of the contact lens were made in air.

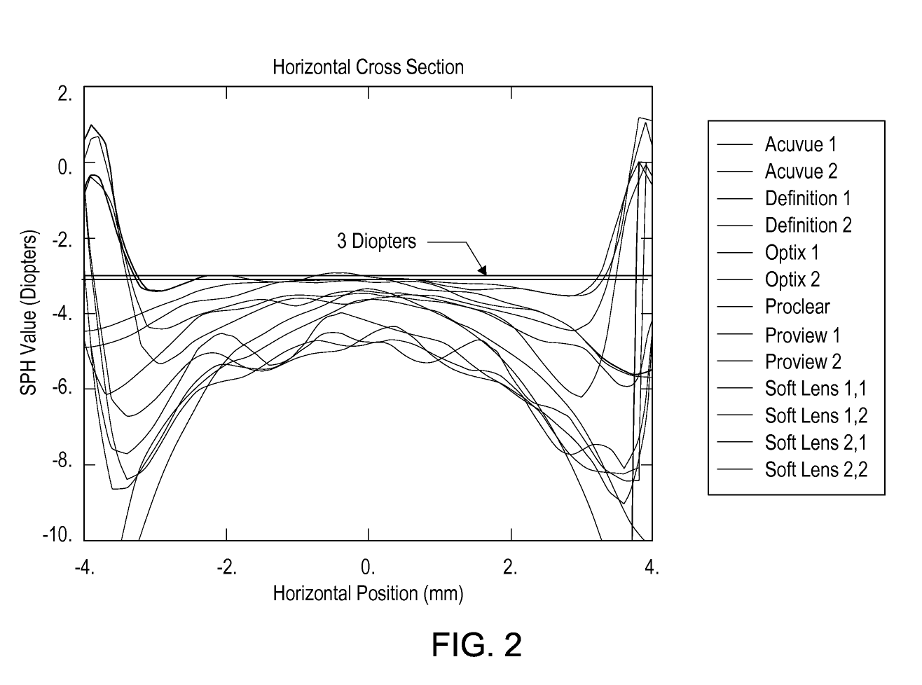

FIG. 2 shows the power profile of 13 commercially available monofocal contact lenses after measurement with suitable optical characterization means described above. From FIG. 2, it is evident that the power profile of these lenses varies considerably, despite that fact that each lens has the nominal power value of −3D.

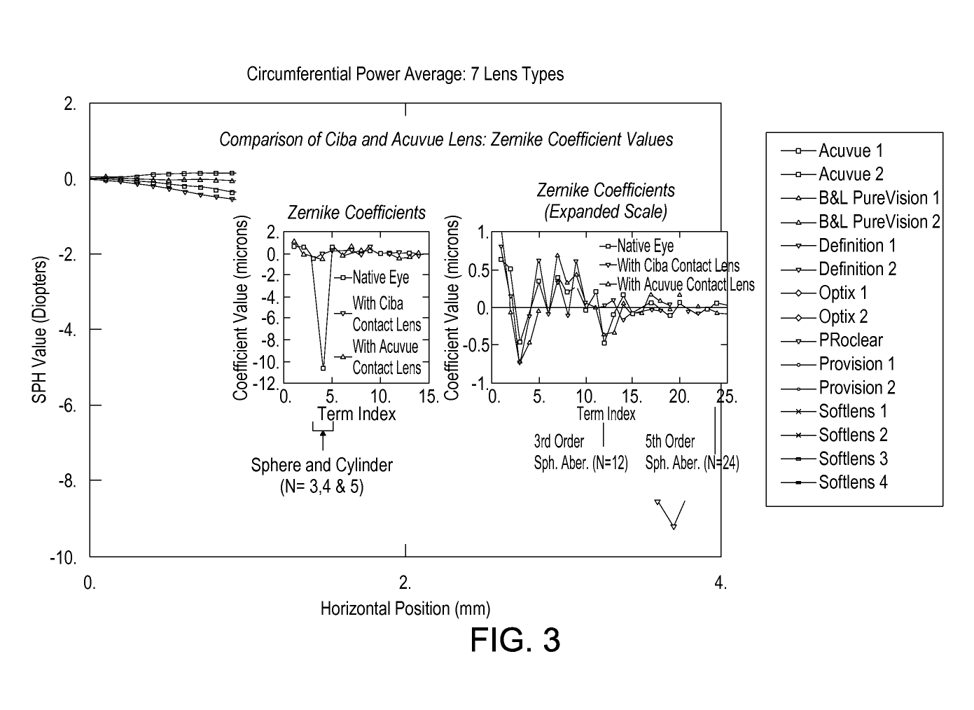

FIG. 3 on the left, shows the degree to which these lenses differ from the nominal −3D power as a function of their horizontal distance from the center of the lens. The middle portion of FIG. 3 shows the wavefront power of the eye without correction and while wearing the CIBA and Acuvue contact lenses. It is evident that lenses of the same nominal power provide different degrees of correction to the eye.

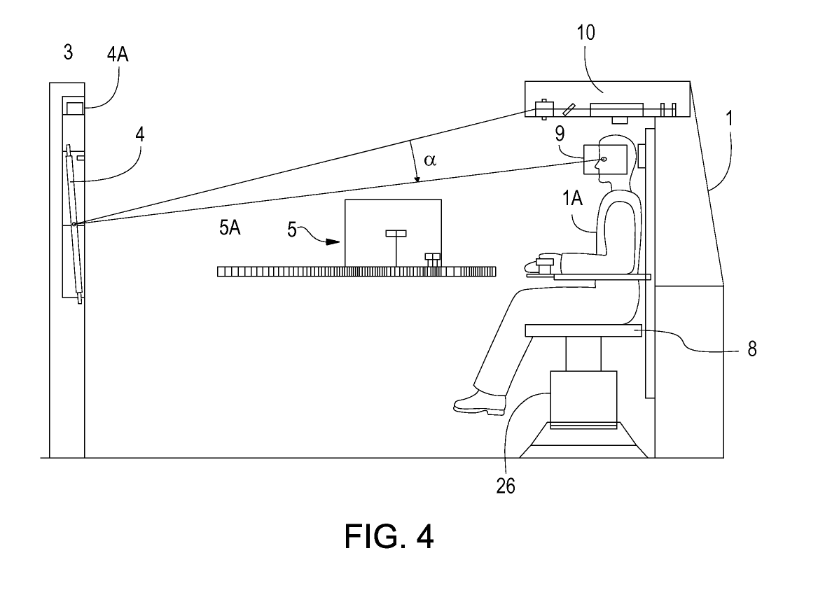

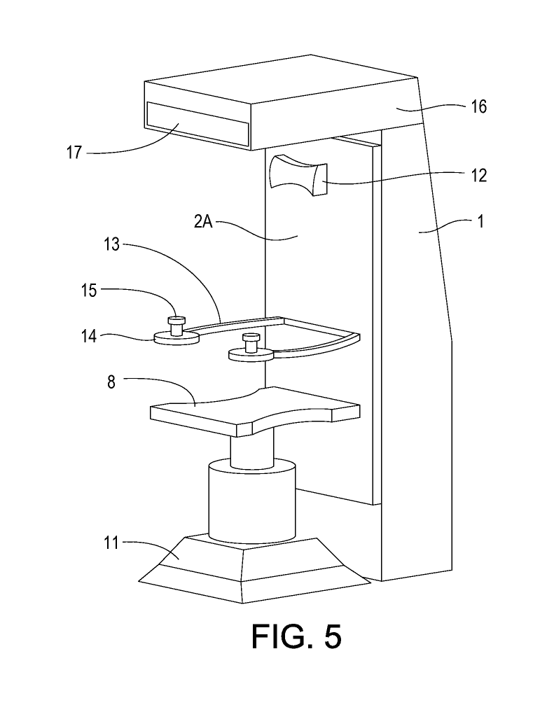

FIGS. 4 and 5 show the contact lens emulation apparatus is comprised of tower 1, an examination chair 2A, a viewport 3 which houses a reflective field mirror 4 and optional camera 4A, and an operator control terminal 5. The patient 1A undergoing vision testing with contact lens emulator is seated in the examination chair seat 8 which is adjusted to place the patient's eyes within the desired examination position noted by box 9. Images are generated by wavefront generators in the wavefront generator 10 and directed to a field mirror 4 in the viewport 3 where they are reflected to the patient's eyes located within the desired examination position 9. Behind the patient, rear cabinet 1 houses a computer, a power supply, and other specialty electronics to control the wavefront generators 10. Images projected from the wavefront generators are reflected by field mirror 4 and viewed by the patient seated in the exam chair 8.

FIG. 4 shows a perspective view of the examination chair 2A of the contact lens emulator that is located adjacent, and forward of, the vertical tower 1, and it is preferentially mechanically isolated from the tower 1 so that patient movements in the chair are not transmitted to the components in the tower. The examination chair has a seat portion 8, the position of which is adjustable through motor means located in the base of the chair 11 that may be made responsive to the system computer. The seat back has a head rest 12 that may be adjustable through manual or by automatic means made responsive to the system computer. Optional head restraint (not shown) may be deployed from the underside of optical tray 10 to aid in stabilizing the patient's head during the exam.

The examination chair has arm rests 13, each of which has a platform 14 for supporting patient input means 15. In one embodiment, the input means is a rotary haptic controller that the patient may rotate, translate, or depress to provide input to the system computer during the examination. Suitable haptic controllers are manufactured by Immersion Technologies, San Jose, Calif. 95131, and such controllers are particularly suited to providing intuitive input to the system during the exam. Numerous other input devices are known, such as a mouse, a joystick, a rotary control, touch-sensitive screen, voice, and other control means, any of which may be employed as alternative embodiments.

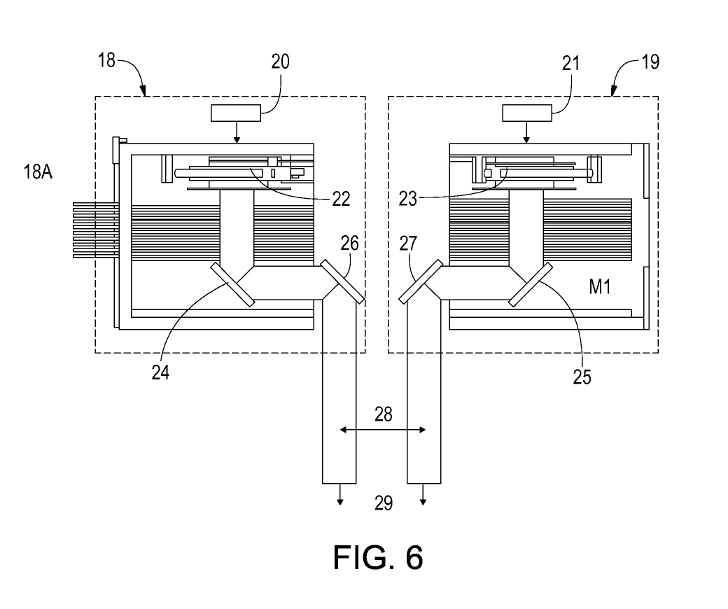

FIG. 6 shows a top view of the wavefront generators for the right eye 18 and left eye 19 with the adjustable lenses and accessory lenses removed. Display means for the right eye 20 and left eye 21 generate images. One suitable image generating means is model SXGA OLED-XL™, made by EMagin Company, Bellevue, Wash. Numerous other image generating means and modalities are known in the art including LED, OLED, DLP, CRT and other means, any and all of which may be suitable for alternative embodiments.

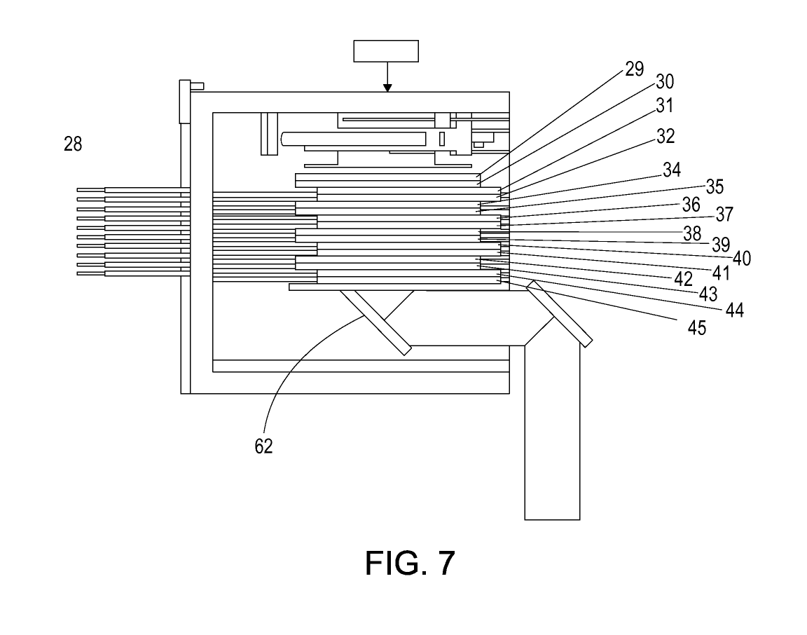

Images generated by 20 and 21 pass through collimating lens 22 and 23. Collimated light of the images then traverses the stack of adjustable optical elements and accessory lens elements, shown in detail in FIG. 7, and described below, where they are redirected by beam turning mirrors 24 and 26 for the right eye, and by beam turning mirrors 25 and 27 for the left eye where they are then directed towards the field mirror 29. The position and angle of turning mirrors 24, 25, 26, and 27 can be made responsive to the system computer by actuator means 18A in order to direct the beam to the field mirror and to adjust the spacing between the left and right beam paths to that of the patient's inter-pupillary distance, 28. In a preferred embodiment, turning mirrors 24, 25, 26, and 27 may be made responsive to an eye and/or gaze tracking system to aid in directing the beam along the desired path for patient testing.

Suitable adjustable lenses for use in the wavefront generators are lenses described by Alvarez in U.S. Pat. No. 3,305,294. In general, theses lenses are comprised of two elements, each surface of which may be described by a cubic polynomial equation and each element is a mirror image of its fellow element. It is known to those skilled in the art that the coefficients of the equations that define the shape of the Alvarez lens elements may be optimized to improve their optical performance and to minimize undesirable aberrations, by, for example, using suitable optical design software such as ZeMax (Radiant ZEMAX LLC, 3001 112th Avenue NE, Suite 202, Bellevue, Wash. 98004-8017 USA). Such modifications of the adjustable lenses are fully envisioned within the scope of the present disclosure.

As the elements of the Alvarez lens pairs are made to translate relative to each other in a direction that is perpendicular to the optical axis of the element, the optical power imparted to an image passing through them changes as a function of the amount of translation. The lenses are mounted in surrounding frames and they are translated by actuator means such as, by example, control cables 18A such that their motion is made responsive to the system computer. Alternate lens actuation means are known in the art and are within the scope of the present disclosure.

Other types of adjustable lenses and mirrors are known in the art that may be used in the wavefront generator to modulate the wavefront of an image and they are considered to be within the scope of the invention. Deformable mirrors that may be made responsive to a computer are known such as those manufactured by Edmunds Optics, 101 East Gloucester Pike, Barrington, N.J. 08007-1380. As an alternative embodiment, the adjustable Alvarez lenses described above may be replaced by fixed lenses, by one or more deformable mirrors, or by any combination of fixed lenses, deformable mirrors, and Alvarez lenses and remain under the scope of the present disclosure. Another embodiment involves the use of one or a plurality of discrete lenses, disposed in a rack or other arrangement, and used to modulate the wavefront of the image.

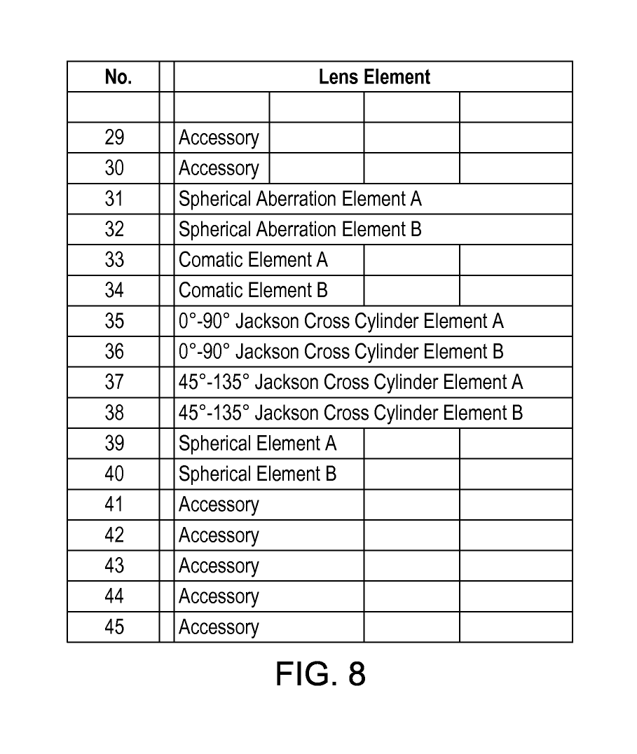

FIG. 7 shows a more detailed view of the wavefront generator for the right eye showing the adjustable Alvarez lens pairs and the accessory lens pairs 29-45 that are used to modify the wavefront of the image that is created by display means 20. The identity of these lenses of one embodiment is listed in FIG. 8.

In general, it is envisioned that the optical elements listed in FIG. 8 will be selected to modulate the wavefront of an image in order to provide a full range of correction of refractive errors with contact lenses from −20D to +20D and astigmatic corrections up to, or beyond, 8D. In addition to providing spherical and cylindrical modulations to the wavefront, the lenses will be able to impart higher order aberrations to the wavefront including spherical aberration and comatic aberrations. As an alternative embodiment, the wavefront generator may utilize fixed and adjustable lens elements to impart spherical and cylindrical modulations to the wavefront, and employ deformable mirror elements to impart higher order aberrations to the wavefront of the image.

Phase plates, such as those prepared by lathing a PMMA or other suitable optical material into the desired shape, may be inserted in accessory slots 29, 30, and 41-45 of the wavefront generator in order to impart additional modulations to the wavefront that are not imparted by the adjustable optical components in order to effectively emulate the wavefront modulation of the contact lens measured by the optical characterization system, D.

FIG. 4 shows a side view of the viewport 3, that houses the field mirror 4. In a preferred embodiment, the field mirror is round in shape and it has a spherical concave curvature with a radius of curvature approximately 2.5M and a diameter between 10″ and 24.″ Such mirrors are known in telescopic applications and a suitable mirror may be procured from Star Instruments, Newnan, Ga. 30263-7424. Alternative embodiments for spherical mirrors are known such as CFRP (carbon fiber reinforced polymer) spherical rectangular mirrors which may be procured from Composite Mirrors Applications in Arizona. Alternative embodiments for the focusing system include the use of an aspheric mirror, a toroidal mirror, a mirror that is non-circular in shape, and a plano mirror.

In a preferred embodiment, the radius of curvature of the mirror 4 corresponds to the approximate distance between the corneal plane of the patient's eyes (at the nominal testing position 9) to the mirror, and from the center of the wavefront generator 10 to the field mirror 4. It is known to those skilled in the art that an object located at a distance from a spherical concave mirror that is equal to the radius of curvature of the mirror, produces an image at a conjugate optical plane of the mirror with a magnification of one. Because the adjustable lenses and the corneal plane are located at optical planes that are conjugate with respect to the field mirror, the adjustable lenses will have the same effective power at the corneal plane as they do in the wavefront generator. Stated differently, the field mirror optically relays the adjustable lenses in the wavefront generator to, or near, the corneal plane, while leaving the space in front of the eye free of physical lenses or other instrumentation.

Operating the instrument at, or near, this condition of “unity magnification” is a preferred embodiment. However, it is known that changes in effective lens power that result from Alvarez lenses imaged at non-unity magnifications may be compensated for by calibration tables and/or by adjusting the adjustable optical elements in wavefront generator 10 to correct for the operation of the device at such non-unity magnifications. Such corrections may be made by the system computer automatically without the input by the operator. It is also known that only one location in the Alvarez stack can be exactly at the center of curvature along the optical axis of the mirror, and that some correction factor(s) must be applied to the lenses in the wavefront generator that are located adjacent the center of curvature.

As shown in FIG. 4, a desk 5A is provided to support the display terminal 5 used by the operator to provide control inputs to the computer and to receive displays from the device. Operator input may be provided by conventional keyboard, mouse, or optional haptic means to control the contact lens emulator during the examination. These devices are connected to the system computer through conventional cable, fiber optic, or wireless means.

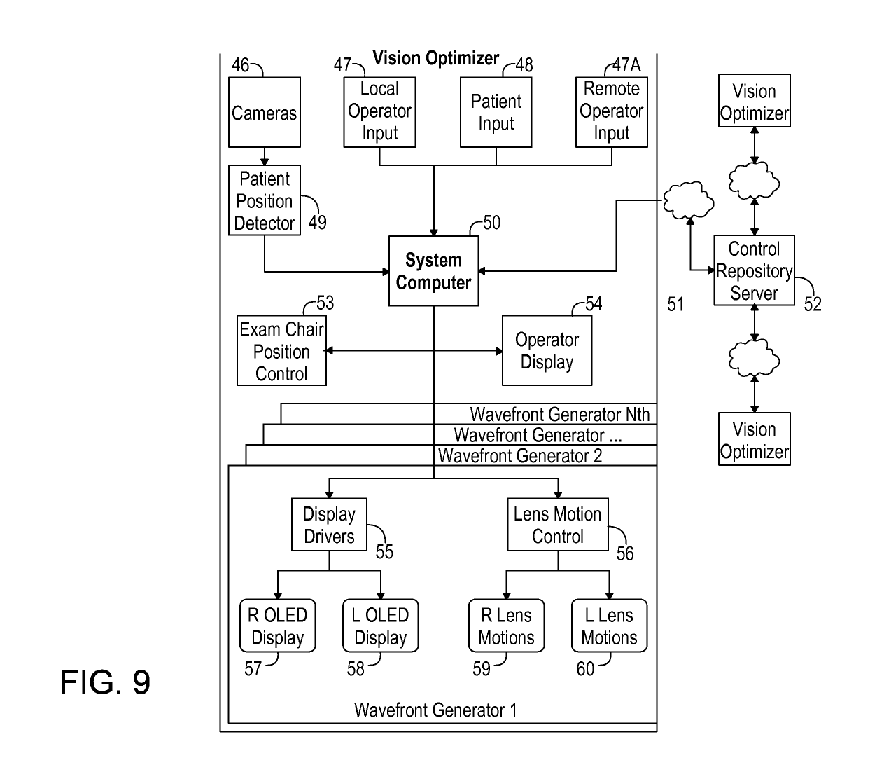

FIG. 9 shows inputs and outputs of the system computer 50 to different subsystems of the apparatus. Camera 46 provides information to the patient position detector 49, which provides input to system computer 50. Operator inputs 47 and patient inputs 48 are provided to the system computer.

The system computer 50 receives inputs and provides outputs to database storage system 52, which in a preferred embodiment may be transmitted through the Internet 51.

The system computer 50 provides outputs to display drivers 55 which run the digital displays 57 and 58, which in a preferred embodiment, may be organic light emitting diodes described above. The system computer 50 provides outputs to lens motion control system 56 which directs the actuators that drive the adjustable lenses for the right and left channels of the wavefront generators, 59 and 60, respectively. The lens motion control 60, also controls the positions of accessory lenses which may include phase plates that may be introduced into one or more of the accessory lens slots of the wavefront generator as shown in 29, 30 and 41-45, and described in more detail below.

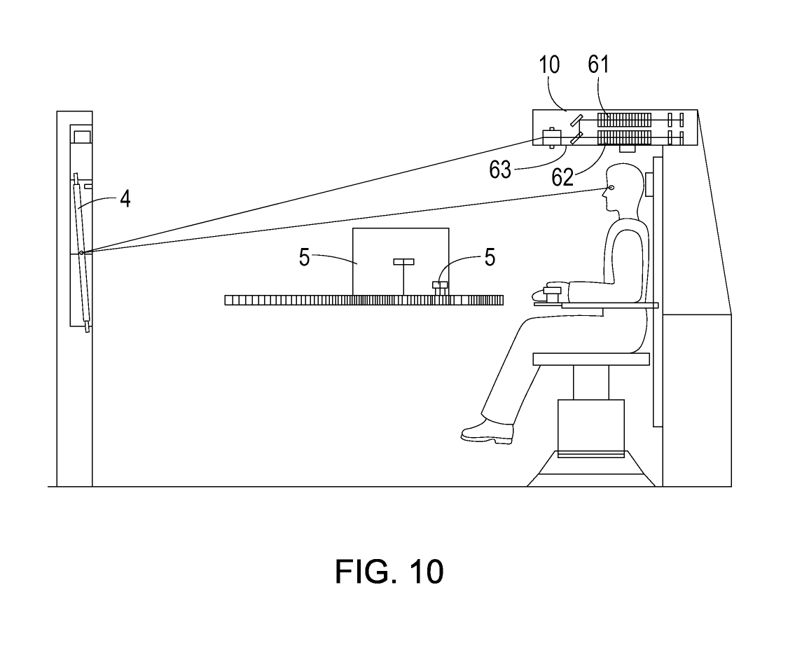

FIG. 10 shows a side view of a preferred embodiment in which two wavefront generators per eye, four total, are housed in the optical tray. For the right eye channels, the images of upper wavefront generator 61 and lower wavefront generator 62 are combined by beam combining element 63 and thereafter directed out the wavefront generator towards field mirror 4. As will be described below, a plurality of wavefront generators per eye allows for patients to view and compare the images produced by the emulated optical properties of contact lenses of a different design on a side-by-side and simultaneous basis.

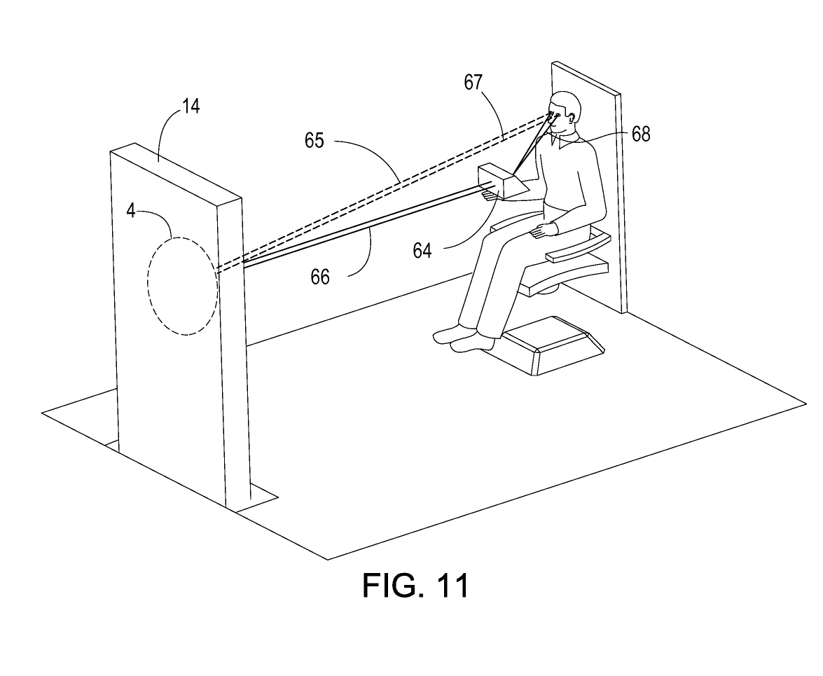

FIG. 11 shows a near-viewing display 64 of the focusing system of the apparatus. When the field mirror 4 is caused to redirect the path of the beam paths from 65 to 66, mirrors (not shown) inside the near viewing display 64 redirect the beams to the patient's eyes along paths 67 and 68. The mirrors cause the images to diverge with respect to each other, and to appear to the patient in the exam chair as if they emerged from the viewing surface 73 of the near viewing display 64.

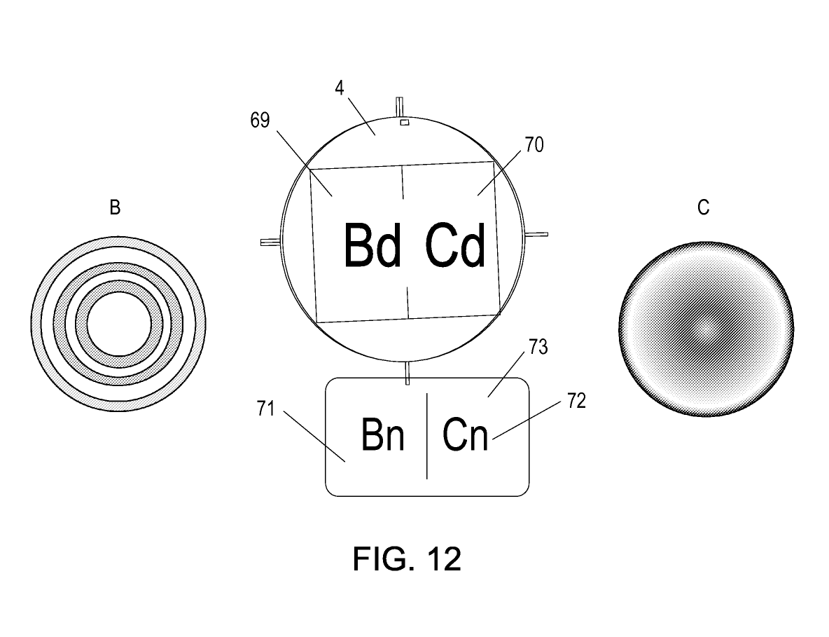

FIG. 12 shows the patient's right eye view of the viewport 4 and the surface of the near viewing display 73. When an embodiment with two or more wavefront generators is employed, the patient is able to preview and compare images with wavefronts emulating that of contact lens B and contact lens C on a side-by-side basis at both near, Bn and Cn, and distance Bd and Cd, viewing distances through the field mirror 4 and the surface 73 of the near viewing display 64.

The use of the apparatus to determine the optical characteristics of a plurality of contact lenses and the emulation of the performance of those contact lenses in a prospective patient will now be described.

Three contact lenses of three different designs are shown as A, B, and C in FIG. 1. The optical properties of each contact lens are measured by contact lens measuring means, and these wavefronts are represented by mathematical functions Eₐ, E_{b}, and E_{c}, respectively. These mathematical functions describe the three dimensional shape of a wavefront of light at a particular distance after it passes through the contact lens. Suitable functions for describing this wavefront include a Zernicke polynomial expansion series, a Fournier function, a Taylor expansion series, or similar mathematical expressions. Optionally, optical properties in addition to the phase change that are imparted by the contact lenses can be measured, for example, the transmission of light as a function of wavelength and this information can be used to increase the fidelity of the emulation of the contact lens by the wavefront generator.

Next, it is necessary to determine if the aspheric powers E′ₐ, E′_{b}, and E′ of the contact lenses can be emulated by the adjustable optical components of the wavefront generator that are listed in FIG. 8. In general, for contact lenses of refractive designs that have contiguous power transitions, it will be possible to emulate the contact lens power with a combination of adjustable Alvarez lenses and deformable mirrors. However, for diffractive contact lenses that employ Fresnel optics and for refractive designs that have abrupt changes in optical power between zones, it may be necessary to procure a phase plate of PMMA or other suitable optical material that, when placed in series with the adjustable optical elements in the wavefront generator will result in an accurate emulation of the optical properties of the contact lens.

In general, the shape of the phase plate required can be determined by subtracting the closest-fit wavefront that can be generated by the adjustable lenses listed in FIG. 8 from the residual aspheric power of the contact lens measurement, for example, E′ₐ, E′_{b}, and E′_{c}, described above.

Once the necessary phase plate(s) has been procured (if needed) for the contact lenses to be emulated, the emulation of the contact lens in a prospective patient may proceed as described above.

In an alternative embodiment, the actual contact lens to be emulated is placed into the wavefront generator by placing it in an appropriate containment holder and interposing it in the wavefront generator in the appropriate location, such as accessory slot 29 shown in FIG. 7. Various embodiments of the apparatus allow placing the contact lens in air or in a suitable fluid.

The adjustable optical elements in the wavefront generator are interposed in the beam path to emulate the optical properties of the contact lens as if the contact lens were placed on the cornea of the eye.

When an image produced by image generation means 20 traverses the wavefront generator 18, and is focused by the field mirror 4, it will appear to the patient as if the image passed through the contact lens when the contact lens is placed on the patient's cornea. Stated differently, to a patient viewing a distant object in mirror 4, the object would appear as if light rays from the object passed through the contact lens when being worn on the patient's cornea.

Assessing the quality of vision for both near, distant, and intermediate viewing distances is desirable for patients to evaluate the performance of different contact lenses in managing the patient's presbyopia.

FIG. 11 shows the patient viewing near images in the apparatus. For near-image viewing, field mirror 4 is tilted down in order to re-direct the light beams from paths 65 to paths 66, which causes them to pass through near-viewing assembly 64.

For near viewing, the adjustable spherical lenses in the wavefront generators 18 and 19 are adjusted to impart the appropriate divergence to the wavefront of the image that is associated with the near viewing distance. For example, to properly emulate the viewing of an image that emerges from the viewing surface 73 of near viewing assembly 64 when it is located 25 cm from the patient's eyes, approximately −4D of spherical lens power would be added to the pre-existing settings of the adjustable optical elements in the wavefront generator and this −4D of divergence is then optically relayed to the patient's spectacle plane by the field mirror as described above. To the patient, it will appear as if the image is emerging from the surface 73 of the near viewing assembly.

In a preferred embodiment, the field mirror 4 is made responsive to an eye and gaze tracking system which receives inputs from camera(s) 4A. When the eye and gaze tracking system detects that the patient's gaze is directed downward to the viewing surface 73 of the near viewing assembly 64, the field mirror 4 is tilted downward so that it redirects the beams from paths 65 to 66, thereby causing them to pass through the near viewing assembly 64.

FIG. 12 shows the patient's right eye view of the field mirror 4 and the near viewing surface 73 of the near viewing assembly 64. The wavefront generator 61 is producing image B through the necessary combinations of optical elements required to emulate the optical properties of contact lens B, and wavefront generator 62 is producing image C through the necessary combinations of optical elements required to emulate the optical properties of contact lens C.

Thus, the patient can preview, compare, and select the contact lens optics of either contact lens B or contact lens C that provides the best quality of image. These images may be compared simultaneously or substantially simultaneously on a side-by-side basis. Similarly, when viewing the near viewing surface 73, images A and B are produced in a similar fashion by redirecting field mirror 4 and by adjusting the adjustable lenses in the wavefront generator to generate the appropriate divergence of light for the viewing distance of viewing surface 73 of the near viewing assembly 64. Thus a plurality of contact lenses are or may be emulated simultaneously or perceived simultaneously by the patient.

By activating the wavefront generators for the left eye, a binocular comparison of images B and C can be attained in a similar fashion.

The disclosure above provides many useful inventive features over prior art methods.

Means are provided to characterize the optical properties of any contact lens, and to accurately emulate those optical properties in a prospective contact lens patient under realistic viewing conditions over near, intermediate, and far away distances. This allows the prospective contact lens patient to preview, compare, and select a particular contact lens design that they prefer based upon the patient's subjective appraisal.

Unlike prior art methods, the present apparatus and method provide the ability to compare the performance of different contact lens designs over a variety of viewing distances under natural viewing conditions free of obstructing optical instrumentation and other limitations of the prior art. Since a major benefit of a presbyopia-correcting design is to provide clear vision over the typical range of viewing distances, the device provides a useful means for the patient to test the performance of the contact lens design over the full range of viewing distances that the patient requires.

Another novel feature of the present apparatus and method is its ability for patients to assess the performance of various contact lens designs over a range of image illuminations, colors, and contrasts. By adjusting the output of the image projectors, patients can see how the contact lens designs compare as illumination and contrast rises or falls and as colors change. No prior art method offers this ability.

The novel capabilities provided by this device will allow doctors to determine which patients are good candidates for a presbyopia correcting contact lens, monovision contact lenses, and other types of contact lenses, etc., and which are not, and it will provide information that is useful to select the particular type of contact lens that is most likely to provide the patient with the most satisfactory visual outcome.

Another novel feature is the ability to stabilize the image into the appropriate image plane by using an eye and gaze tracker. This relieves the patient of the need to hold still during the test and it facilitates a more realistic emulation of contact lens performance under natural viewing conditions. The testing is also done with no instruments or other visual obstructions in the patient's filed of view, unlike prior art methods and devices. Optical parameters used to manufacture or select contact lenses can be determined in much higher resolution increments, such as 0.01D, as opposed to the 0.25D increments used in prior art methods. As contact lens manufacturing techniques and methods have improved, the present methods and apparatuses now provide the means to prescribe and/or to custom-manufacture lenses that provide patients with vastly improved eyesight compared to contact lenses prescribed using various prior art methods. Similarly, this disclosure provides practitioners the means to improve visual function with contact lenses that include prescription for the correction or induction of higher order aberrations.

While a method and apparatus for vision testing in order to provide vision corrective contact lenses to a patient, and modifications thereof, have been shown and described in detail herein, various additional changes and modifications may be made without departing from the scope of the present disclosure or the appended claims.

We claim:

1. A method of vision testing that allows a patient to preview the optical properties of an contact lens to correct the patient's vision, comprising the steps of:
a. determining the optical properties of a contact lens to be emulated;
b. generating a static or dynamic (movie) image viewable by a patient;
c. modulating the wavefront of the image to produce an image on the patient's retina that emulates the image that would result when the contact lens is placed on the patient's cornea

2. The method of claim 1 in which modulating the wavefront of the image is performed remotely from the patient.

3. The method of claim 1 in which a plurality of contact lenses are emulated simultaneously or perceived simultaneously by the patient.

4. The method of claim 1 in which said modulating step includes interposing a contact lens in said wavefront generator and projecting said image through said contact lens.

5. The method of claim 1 in which said modifying step is responsive to inputs provided by the patient.

6. The method of claim 1 in which a second wavefront generator emulates an image on the patient's retina produced by a second contact lens so as to allow the patient to compare the two images in a substantially simultaneous manner.

7. The method of claim 1 in which a plurality of wavefronts emulating a plurality of contact lenses are produced on the patient's retina so as to allow the patient to compare and select a preferred image.

8. A vision testing apparatus that allows a patient to preview, compare, and select the optical properties provided by a contact lens, comprising:
means for measuring or inputting the optical properties of a contact lens
means for projecting an image from a wavefront generator through optical elements that are under control of a computer;
means for focusing said image to a position optically conjugate to the patient's eye;
and
means for adjusting the wavefront generator to produce an image on the patient's retina that emulates the image that would result when the contact lens is worn on the cornea.

9. The apparatus of claim 8 in which said means for modifying the modulation of the wavefront of the image is responsive to input provided by the patient.

10. The apparatus of claim 8 including one or more contact lenses that are interposed in said wavefront generator for projecting said image through said one or more contact lenses to produce multiple images viewable by the patient.

11. The apparatus of claim 8 including one or more contact lenses interposed in said wavefront generator for projecting said image through said contact lenses.

12. The method of claim 8 including input means for adjusting of the wavefront generator image in response to input from the patient.
